(19) United States
(12) Patent Application Publication
(10) Pub. No.: US 2004/0116527 A1
(43) Pub. Date: Jun. 17, 2004

(54) Agents against stress-induced diseases

(75) Inventors: Miroslav Smriga, Kawasaki-shi (JP); Hisayuki Uneyama, Kawasaki-shi (JP); Kunio Torii, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO., INC., Tokyo (JP)

(21) Appl. No.: 10/667,783

(22) Filed: Sep. 23, 2003

Related U.S. Application Data
(63) Continuation of application No. PCT/JP02/02571, filed on Mar. 19, 2002.

(30) Foreign Application Priority Data
Mar. 23, 2001 (JP) 2001-085800
Dec. 14, 2001 (JP) 2001-382190

(51) Int. Cl. A61K 31/198

(52) U.S. Cl. 514/564

(57) ABSTRACT

The present invention relates to an agent for treating and/or preventing stress-induced diseases, and a pharmaceutical composition (pharmaceutical product), a food, drink, or a feed containing the same. More specifically, the present invention relates to an agent (pharmaceutical drug) containing lysine as the active (effective) ingredient, and a pharmaceutical product, a food, a drink, or a feed containing the agent (pharmaceutical drug). Furthermore, the present invention relates to a method for suppressing stress, and a method for the therapeutic treatment, amelioration and/or prevention of stress-induced diseases, as well as a use of lysine for anti-stress agents or stress suppressor.

### CROSS-REFERENCE TO RELATED APPLICATIONS

The present application is a continuation of PCT International Application PCT/JP02/02571, filed on Mar. 19, 2002, and claims priority to Japanese Patent Application No. JP 2001-085800, filed on Mar. 23, 2001, and Japanese Patent Application No. JP 2001-382190, filed on Dec. 14, 2001, each of which is hereby incorporated by reference in their entirety.

### BACKGROUND OF THE INVENTION

1. Field of the Invention

The present invention relates to an agent for treating and/or preventing stress-induced diseases, and a pharmaceutical composition (pharmaceutical product), a food, drink, or a feed containing the same. More specifically, the present invention relates to an agent (pharmaceutical drug) containing lysine as the active (effective) ingredient, and a pharmaceutical product, a food, a drink, or a feed containing the agent (pharmaceutical drug). Furthermore, the present invention relates to a method for suppressing stress, and a method for the therapeutic treatment, amelioration and/or prevention of stress-induced diseases, as well as a use of lysine for anti-stress agents or stress suppressor.

2. Discussion of the Background

Lysine is one of the essential amino acids, which is commonly provided in the cereals we eat. However, lysine is the amino acid that most people's diet is deficient in. People that mainly eat rice, including Japanese people, have evaded lysine deficiency by eating wheat and beans containing lysine, without much ingestion of animal foods. However, people that primarily eat a diet consisting of corn lapse into lysine deficiency, when they do not eat or drink milk or cattle meat.

With the ever-expanding global population, agricultural production cannot keep pace and, therefore, it is assumed that lysine deficiency may occur mainly in Asia (see Kunio Torii, Clinical Nutrition (Rinsho-Eiyo), 1997, 90(3), 229-232). Despite the seeming misnomer in which the modern age is called the “well-fed era”, an increased relationship between diseases and an unbalanced nutritional intake due to an unbalanced diet in aged people and too low dietary intake of juvenile and young men and women has gained notoriety. Currently, no detailed survey exists over the nutritious state of people included in those groups and the diseases by which those people are readily affected. It can easily be speculated that those people readily fall into lysine deficiency.

During the course of investigations in terms of nutrition science and health science relating to amino acid, the inventors experimentally found that so-called stress-induced diseases were caused by a lysine deficiency such that resistance against stresses from outer environment was significantly reduced. It was found by using for example the elevated T-maze test of Graeff, et al. (see Graeff F. G. et al., J. Med. Biol. Res., 1993, 26, 67-70) that the anxiety symptoms in rats at lysine deficient state were exacerbated. Using a test example in a water-immersion restraint and stress model, it was confirmed that the symptom of gastric ulcer in rats at lysine deficient state was significantly exacerbated.

In such a current complex social environment, on the other hand, it is confirmed that the incidence of stress-induced diseases is high because sensitivity to stresses and the like from outer environment is also increased even at nutritious state with no lysine deficiency.

Stress-induced diseases mean organ disorders of brain (mind) per se or peripheral organs via the affection of the autonomic nervous system or the endocrine system with psychological or physical stressful stimulants (stressors) directly or via the brain emotional system. The relation between stress-induced diseases and actual diseases has not yet been accurately elucidated. In the present application, however, stress-induced diseases include neurosis such as anxiety disorders (panic disorders and general anxiety disorders), psychosomatic disorders (somatopathy), dissociated disorders and emotion disorders, and diseases due to psychological stresses, such as abnormal motions of gastric organs (digestive organs) (abnormal gastric (digestive) motion), irritable colon syndrome and gastric (digestive organ) ulcer and additionally includes circulatory disorders such as arrhythmia, angina pectoris, and hypertension, immune disorders such as functional abnormality of lymphocyte, hyperphagia and neurological sitophobia, psilosis, d impotency and the like. Furthermore, those to be classified in to the group of psychosomatic diseases are all included therein alike.

As pharmaceutical products for therapeutically treating these stress-induced diseases, a great number of anti-anxiety agents (benzodiazepine derivatives and the like), anti-depression agents (monoamine uptake-inhibiting agents, tricyclic pharmaceutical drugs and the like), and nosotropic pharmaceutical drugs for organic lesions of peripheral organs (for example, antacids, protective agents of gastric mucus, acid secretion suppressors and the like in case of gastric ulcer) have been developed to reduce psychological stressors. Although these pharmaceutical products exert certain effects, their effects essentially involve dependency and side effects. Therefore, these pharmaceutical products have not yet produced radical treatment of such diseases. For nutritional improvement, addition of calcium and certain types of vitamins has been attempted. However, no definite therapeutic results have been achieved. Currently, not any agents (pharmaceutical drugs) (pharmaceutical products), foods or drinks, feeds and the like capable of preventing the onset of these stress-induced diseases has been developed yet.

In such circumstances, an agent (pharmaceutical drug) used effectively for stress-induced diseases (agents against (for opposing) stress-induced diseases) and an agent (pharmaceutical drug) capable of preventing these diseases in particular are now in need. In particular, the agent should capable of being incorporated (ingested) safely and can be widely used in pharmaceutical products, foods or drinks, feeds and the like.

In recent years, additionally, clinical practice has paid significant attention to the improvement of quality of life (QOL). Excess psychological stress deteriorates not only the QOL of individual lives after diseases but also the QOL of their daily lives. Such QOL-improving agents for the purpose of improving QOL or agents for preventing such diseases have been demanded strongly in recent years. As a result, numerous ventures have been initiated to develop agents for treatment and/or prevention of stress-induced diseases. However, none of these companies have achieved the development of a safe agent for the radical therapy thereof.

The effects of pharmaceutical products, foods and/or drinks and feeds on stress-induced diseases, particularly the effects thereof for preventing, ameliorating and therapeutically treating stress-induced diseases can be evaluated in experiments in model rats. The effects on anxiety disorders can be evaluated in models for evaluating anxiety symptoms, using the elevated T-maze test also for use in the evaluation system of anti-anxiety agents. Irritable colon syndrome can be evaluated in wrap stress resistant model (WRS). Further, the evaluation method using water-immersion restraint rat can be applied for assessing the effects on gastric (digestive organ) ulcer. These evaluation methods are widely used as approaches for evaluating the pharmaceutical efficacies of pharmaceutical products for the gastric system (systema digestorium) (see Graeff F. G. et al., J. Med. Biol. Res., 1993, 26, 67-70; Graeff F. G. et al., Pharmacol. Biochem. Behav., 1996, 54, 129-141; Ito C. et al., J. Pharmacol. Exp. Ther., 1997, 280(1), 67-72; Kishibayashi N. et al., Jpn. J. Pharmacol., 1993, 63, 495-502; Takenaka H. et al., Planta. Med., 1993, 59, 421-4; Itoh Y. et al., Digestion 1991, 48, 25-33: Tanaka T. et al., Arzneimittelforschung 1993, 43, 558-62).

As one of markers indicating the onset of stress-induced diseases, the neurotransmitters serotonin has been known. Serotonin is now drawing attention as one of neurotransmitters that generates emotion in a site responsible for emotion in the brain (the amygdale). It is experimentally verified that the elevation of the serotonin concentration in the amygdala in animals, such as rat, lowers the threshold against anxiety, such that anxiety symptoms are induced. In contrast, reduction of serotonin concentration reduces anxiety-like symptoms at experiments (see Gardner C. R., Pharmacol. Biochem. Behav., 1986, 24, 1479-85; Chung et al., Neuroscience, 2000, 95, 453-63; Kilt et al., Psychopharmacology (Berl), 1981, 74, 290-6). Additionally, a report tells abnormalities in the serotonin system in brain in experimental stress-induced gastric ulcer model (see Hellhammer et al., Psychosom Med., 1983, 45, 115-22). It is confirmed that pharmaceutical drugs with actions antagonistic to serotonin produce therapeutic effects in stress-induced gastric ulcer model and irritable colon syndrome model (see Mertz H R, Curr Gastroenterol Rep., 1999, 1, 433-40; Camilleri M., Am. J. Med. 1999, 107, 27S-32S; Erin N. et al., Peptides, 1997, 18, 893-8). Based on these findings, it is suggested that the measurement of the serotonin concentration in brain is effective as the approach for evaluating the effect on the prevention of stress-induced diseases.

It has been known that various stresses on animals during feeding and housing cause problems such as the deterioration of culture results in the fields of cattle-raising industries (livestock industries), fishery and culture industries and the like.

1. Stress from High-Density Housing

So as to elevate productivity, generally, chicken, pig, fish and the like are fed and housed at a high density. The higher density for feeding and culture causes stress, involving the decrease of the intake of feed, the reduction of immune potency and the like. For example, Nippon Feeding and Culture Standards (for chicken (poultry)), the 1997 version describes on page 60 as follows.

Excessively densified feeding and culture cause stress in chicken, involving the decrease of the productivity, the increase of egg break ratio, the occurrence of cannibalism and the decrease of the survival ratio. Therefore, care should be taken.

2. Stress from Weaning

So as to elevate productivity, early weaning is done during the feeding and culture of a pig. Weaning switches the feed to solid feeds, which causes stresses and involves problems such as the decrease of feed intake.

3. Stress During Transfer and Shipping

Stress from the transfer of the place to be fed and cultured causes the decrease of feed intake. Additionally, the stress from the transfer for shipping causes problems such as the deterioration of meat quality.

These stress problems in the cattle industry and fishery and culture are so serious but not any other approaches except for an approach to improve their feeding environment is found as the method for solving these problems. Currently, therefore, not any approach exists from the standpoint of feed.

As described above, substances with actions against stress-induced diseases are needed, which are applicable as pharmaceutical products and foods or drinks. Even for feeds, additional ingredients with an anti-stress effect on such stresses as described above are also in high demand, specifically, ingredients that are to be blended in feeds.

In addition, to provide broiler as a food material of high quality in an inexpensive manner as much as possible, the number of broilers under feeding per feeding area should be elevated, while the feeding efficiency should also be elevated simultaneously. However, feeding of territory-conscious broilers at a certain density or more gives strong stress to the broilers, inducing the decrease of feed intake (so-called stress-induced appetite loss). Additionally, the stresses from feeding at high-density triggers fighting instinct, so that the chickens injure each other (a phenomenon called so-called cannibalism). Consequently, the body weight increase is delayed, involving the reduction of feed efficiency and therefore the deterioration of meat quality, so that the value as a food material is reduced. The problem of the stress from feeding at high density is more serious under very hot conditions, in particular.

From the standpoint of animal welfare, in recent years, it is demanded that feeding animals for food materials should be fed and kept at a state with stress reduced as much as possible, until the animals are sacrificed to death for meat for food. As the effects on humans of pharmaceutical drugs (antibiotics such as penicillin and a certain type of hormone agents) remaining in meat for food have been elucidated gradually, it is more difficult to formulate synthetic pharmaceutical products with stress-reducing actions for feeding animals, which are to be provided in future as food materials. The inventive technique is industrially very useful in that the use of amino acids (lysine+arginine) can elevate the feeding density of broiler without any occurrence of the problem of the stress from feeding at high density, to provide safe food materials at high productivity to the world.

### SUMMARY OF THE INVENTION

It is an object of the present invention to provide pharmaceutical composition for preventing, ameliorating, progress blocking, or therapeutically treating one or more stress-induced diseases and a method of preventing, ameliorating, progress blocking, or therapeutically treating one or more stress-induced diseases by administering the same.

The above objects highlight certain aspects of the invention. Additional objects, aspects and embodiments of the invention are found in the following detailed description of the invention.

### BRIEF DESCRIPTION OF THE FIGURES

A more complete appreciation of the invention and many of the attendant advantages thereof will be readily obtained as the same becomes better understood by reference to the following Figures in conjunction with the detailed description below.

FIG. 1 depicts the time duration how long rats stayed in the box and the number of searching actions, in Example 1.

(Lysine) +: lysine-added diet; −: low lysine diet.

(Stress) +: under stress loading; −: no stress loading

*: p<0.05 vs. lysine-added diet/no stress loading and vs. lysine-added diet/under stress loading.

FIG. 2 depicts the change of serotonin concentration in rat brain (amygdala) in Example 1.

Solid circle: serotonin release from rat on low lysine diet

Open circle: serotonin release from rat on lysine-added diet supplemented with lysine deficiency

*: p<0.05; **: p<0.01 vs. lysine-added diet

FIG. 3 depicts the number of feces excretion and the weight of feces from rats after wrap stress loading in Example 2.

FIG. 4 depicts the area of gastric bleeding in rats and the photopicture thereof in Example 3.

(Graph) Solid square: lysine-added diet; open square: low lysine diet

(Photopicture) A) lysine-added diet; B) low lysine diet

FIG. 5 depicts the changes of total feed intake and body weight over time (days) in rats in Example 4.

Solid square: control diet (normal diet); open square: lysine-added diet

FIG. 6 depicts the results of experiments on stress-induced gastric ulcer in rats in Example 5.

FIG. 7 depicts the results of experiments on the time duration of searching action in rats in Example 6.

FIG. 8 depicts the results of feeding at high density in broilers in Example 8.

Solid square: feeding at normal density; open square: feeding at high density; *: p<0.05 vs. control group.

### DETAILED DESCRIPTION OF THE INVENTION

Unless specifically defined, all technical and scientific terms used herein have the same meaning as commonly understood by a skilled artisan in biochemistry, cellular biology, molecular biology, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The present invention is based, in part, on the inventor's discovery that stress-induced diseases easily occur due to lysine deficiency arising from food conditions. In addition, the inventors have found that stress-induced diseases frequently occur even in normal subjects without lysine deficiency due to the modern social environment. As the results of further investigations, the inventors have found that immediate lysine supplementation for subjects suffering from a lysine-deficient nutritious states and preliminary lysine supplementation in subjects that are without lysine deficiency are an effective therapy for stress-induced diseases.

Particularly, the inventors have found that lysine supplementation can prevent neuroses such as anxiety disorders (panic disorders and general anxiety disorders), psychosomatic disorders, dissociated disorders and emotion disorders, and abnormalities in gastric motions (abnormal gastric (digestive) motion), irritable colon syndrome (irritable bowel syndrome) and gastric (digestive organ) ulcer and that such lysine supplementation can therapeutically treat and ameliorate the diseases readily, even at the onset. Therefore, the inventors have found that preliminary intake of sufficient lysine can produce the preventive effect and that sufficient lysine intake can produce the treatment and amelioration effects, even at the onset of stress-induced disorders.

The inventors have also found that lysine can produce an effect on (competitive with) the stresses intrinsic to animals, when the level of lysine in feeds is maintained at an elevated level.

Based on these various findings, the invention has been achieved.

In accordance with the invention, lysine is used as an active ingredient in a pharmaceutical drug (agent) for therapeutic treatment, amelioration, prevention of stress-induced diseases. Accordingly, it is an object of the present invention to provide an active ingredient in a pharmaceutical drug for preventing stress-induced diseases, to provide pharmaceutical compositions, foods or drinks or feeds with such pharmaceutical efficacy or effect, using or blending such pharmaceutical drug (agent).

In an embodiment of the present invention is a pharmaceutical composition, a food, a drink, or a feed for the therapeutic treatment, amelioration, prevention stress-induced diseases containing lysine. In an additional aspect, the invention relates to an agent for the therapeutic treatment, amelioration, prevention stress-induced diseases containing lysine (a substance (a composition) with an effect (action) against stress-induced diseases in animals, for use or blending in pharmaceutical products, foods or drinks or feeds or the like).

The lysine used in the present invention may be in a free form, but may also be used in the form of a salt(s), or the form of a mixture(s). As such, the mixture may include one or more additional components, which are individually in a free- or salt-form. As used herein, the free form and its salt form(s) are collectively called “lysine”. Further, the L form is adopted because it can be metabolized in biological organisms.

As used herein, the term “for opposing stress-induced diseases” includes those widely used to oppose stress-induced diseases and includes the prevention, amelioration, progress blocking, and therapeutic treatment of stress-induced diseases. Lysine can preferably be used particularly for the prevention of stress-induced diseases.

Any component containing at least lysine and exerting the pharmacological effects as described above may be satisfactory as the component to be contained in the herein-mentioned agents, foods, drinks, or feeds. Unless other components inhibit the effects of the invention, the components may be contained in such agents, foods, drinks, or feeds.

The “other” components that may be further employed in the present therapeutic compositions include one or more additional amino acids. In a particularly preferred embodiment the therapeutic composition may be used to treat ulcers and/or anxiety and contains one or more additional amino acids. Particularly preferable amino acids include arginine (L-arginine), glutamic acid (L-glutamic acid), and aspartic acid (L-aspartic acid). Lysine and arginine may be used in the form of salts with glutamic acid and aspartic acid.

As described above, lysine may be used in the free form, but may also be used in salt form(s). In this case, any acid may be used for composing such salts with no specific limitation. For example, inorganic acids such as hydrochloric acid, sulfuric acid and carbonic acid, and organic acids such as amino acids including glutamic acid and aspartic acid, acetic acid and acetylsalicylic acid, may be mentioned.

In a preferable embodiment, the therapeutic/pharmaceutical compositions contain lysine as the active (effective) ingredient, which may be in the salt form with glutamic acid, and additionally contain arginine (including the form of salt(s)).

Further, the therapeutic/pharmaceutical compositions may contain inorganic matters such as calcium for use as agents against (for opposing) stress-induced diseases.

Moreover, the therapeutic/pharmaceutical compositions of the present invention may contain a pharmaceutically acceptable carrier or excipient.

The inventive products can be used widely for stress-induced diseases. Because the inventive products have great preventive effects, in particular, the inventive products are preferably incorporated during or before stress loading. Consequently, the inventive products can exert the preventive effects. Even if a stress-induced disease occurs, the inventive products can ameliorate or therapeutically treat the disease very readily.

A subject to which the inventive products can be applied includes but is not limited to any animal needing agents against (for opposing) its stress-induced disease. These subjects are not limited to humans but may also include other animals, particularly cattle and animals for fishery and culture.

It is preferred that the inventive products are effectively incorporated prior to or during stress loading.

As to the feeds, animals are the applicable subjects. For various species of animals, particularly cattle and animals for fishery and culture (for example pig, chicken and culture fish), the inventive products are preferably administered during or before the loading of stresses during, for example, transport or shipping. Furthermore, the inventive products are preferable for stresses in case of feeding or culture at higher densities in small feeding lots or culture lots.

For the prevention of stress-induced diseases derived from lysine deficiency, the inventive products can preferably be administered to such a subject. For stress-induced diseases in which the subject is not deficient in lysine, the inventive products may also be administered to obtain a therapeutic advantage.

The term “stress-induced disease” is the generic name of diseases caused by psychological stressors and includes all of so-called psychosomatic diseases in addition to the various diseases described above.

The various stress-induced diseases described above include anxiety disorders, psychosomatic disorders, dissociated disorders, emotion disorders, abnormalities in gastric motion (abnormal gastric (digestive) motion), irritable colon syndrome (irritable bowel syndrome) and gastric (digestive organ) ulcer.

Lysine is preferably incorporated in the inventive therapeutic composition at an amount sufficient to enable an intake of the lysine of approximately 0.001 to 13 g/kg/body weight daily, preferably approximately 0.01 to 6.5 g/kg/body weight daily on a lysine free form basis.

Since lysine can be incorporated from sources other than the inventive products, the lysine intake from the inventive products is preferably controlled in view of the total lysine intake. In this case, lysine is preferably incorporated from the inventive products so that the total lysine intake may be approximately 0.001 to 13 g/kg/body weight daily, preferably approximately 0.01 to 6.5 g/kg/body weight daily on a free form basis.

The daily lysine intake described above is commonly applicable to pharmaceutical products, foods or drinks and feeds. As described below concerning the upper limit, less lysine intake than the daily lysine intake is recommended, which is applicable only to pharmaceutical products or foods or drinks for humans.

The total intake of the lysine may be approximately 0.001 to 1.0 g/kg/body weight daily, preferably approximately 0.01 to 0.5 g/kg/body weight daily on a free form basis. The intake is effective particularly for lysine-deficient humans.

Since lysine can be incorporated from sources other than the inventive products, the lysine intake from the inventive products is preferably controlled in view of the total intake of lysine. In this case, the lysine is preferably incorporated from the inventive products, so that the total intake of lysine may be approximately 0.001 to 1.0 g/kg/body weight daily, preferably approximately 0.01 to 0.5 g/kg/body weight daily on a free form basis.

Any content of the lysine in pharmaceutical products, foods or drinks or feeds is satisfactory, with no specific limitation. Depending on the type or form of such product, the content of the lysine can be selected appropriately, which is approximately 90 to 0.1% (by weight), preferably approximately 10 to 1% (by weight) on a free form basis.

Any form of the inventive products is satisfactory with no specific limitation. In case of pharmaceutical composition, for example, granules, tablets, infusions, injections and the like are selected. In case of foods or drinks, for example, forms of drinks, nutritious agents and health foods in addition of granules and tablets can be suggested. In case of feeds, forms of lysine applicable to feeds are satisfactory, including for example lysine mixed with feeds of normal forms.

As the other components described above, further, inorganic matters such as vitamin and/or calcium can be added and used, on a needed basis.

In an additional aspect, the invention relates to a method for suppressing stress, as well as prevention, amelioration, progress blocking, and therapeutic treatment of stress-induced diseases, by ingestion of lysine or administration into biological organism(s) (living body(ies)). The lysine may be in the form of salt(s).

As to the mode for the incorporation or administration, the lysine may be incorporated or administered in any form of various pharmaceutical compositions, foods or drinks, feeds and the like in accordance with the invention (including the agent against (for opposing) stress-induced diseases).

In a further aspect, the invention relates to a use of lysine for anti-stress agent or the production thereof. The lysine may be in the form of salt(s).

The anti-stress agent (pharmaceutical drug(s) used for stress-induced diseases, which include agent(s) against stress-induced diseases and the preventive agent(s) therefor) is just as described above. As described above, additionally, the mode of any one of the various pharmaceutical compositions, foods or drinks, feeds and the like in accordance with the invention (including the above described agent(s) against stress-induced diseases) or the mode used for them can be listed as the preferable example.

The mode for carrying out the present invention is now described below.

As described above, in one embodiment, the invention relates to lysine-containing pharmaceutical compositions, foods or drinks or feeds for opposing stress-induced diseases, while in an additional embodiment, the invention relates to lysine-containing agents for prevention, amelioration, progress blocking, and therapeutic treatment of stress-induced diseases.

Thus, the invention encompasses pharmaceutical compositions, foods, drinks, feeds and agent against stress-induced diseases, each of which contain lysine as an active (effective) ingredient. For the purpose of giving any pharmacological effect, for example for the purpose of the prevention, amelioration, progress blocking, and therapeutic treatment of stress-induced diseases, the embodiments are used in common. Because the type and form of a final product varies, the difference in terms of these variations should be considered. Unless otherwise stated concerning the difference, however, descriptions in this specification (description) are done in common to the inventions of the four types.

The subject to which the inventive products are given (via eating and drinking, and administration and the like) includes but is not limited to any of such various animals as described above (humans, cattle, animals for fishery and culture, and other animals with a possibility of the onset of stress-induced diseases), which is in need of prevention, amelioration and/or therapeutic treatment and/or the like of stress-induced diseases. Generally, however, the inventive products are applied to mammals, particularly humans (for the feed, the subject includes animals, particularly cattle and animals for fishery and culture (fishes and the like)).

The onset of stress-induced diseases due to lysine deficiency may possibly arise from a diet that consists predominantly of corn with a low content of lysine and at a low level of lysine intake from other foods is continued. In case of unbalanced dietary life in aged people and excessively unbalanced nutrition due to extremely low dietary intake in juvenile and young people, they fall into lysine deficiency. Thus, it is anticipated that they readily cause the onset of stress-induced diseases. For people on such a diet, lysine of preferably approximately 0.001 to 1.0 g/kg/body weight, more preferably approximately 0.01 to 0.5 g/kg/body weight as daily intake on a free form basis is formulated into dosage forms such as granules and powders and is then given (administered), so that the onset of stress-induced diseases can be prevented effectively.

In an environment with no essential lysine deficiency, further, sufficient intake of lysine from the inventive products prior to stress loading or while in a stressful environment can give the preventive effect. Even at the onset of stress-induced diseases, the stress-induced diseases can readily be cured. Then, the inventive products may be given so that the total intake of lysine in that case may be preferably approximately 0.001 to 1.0 g/kg/body weight, more preferably approximately 0.01 to 0.5 g/kg/body weight daily on a free form basis.

For feeds for animals, the upper limit (for humans) of the amount of lysine for use therein can be elevated, while the lower limit thereof remains as it is. The range of preferable numerical figures for feeds is shown below.

On a free form basis, lysine is given at an amount of preferably approximately 0.001 to 13 g/kg/body weight, more preferably approximately 0.01 to 6.5 g/kg/body weight daily on a free form basis. When lysine is given from other sources, lysine is blended in feeds, so that the total intake of lysine may be preferably approximately 0.001 to 13 g/kg/body weight, more preferably approximately 0.01 to 6.5 g/kg/body weight daily on a free form basis. The resulting feeds may satisfactorily be given to intended animals.

Compared with the conventional amount of lysine in blend in feeds, the content of lysine in blend in feeds is preferably fairly high so as to securely attain the intended effects sufficiently. It is recommended that lysine for example at an amount 1.1-fold to 3.0-fold the recommended nutritious requirement of lysine (see NRC, the Nippon feeding standards and the like) is to be blended. Even in this case, lysine may be used in the form of salt(s).

The most appropriate lysine intake currently proposed varies, depending on the animal species and the growth stage thereof. For example, the lysine intake calculated on the basis of the recommended value by the National Research Council (NRC), USA is as follows.

Pig: 790 mg/kg/day (for 5-10 kg body weights); 160 mg/kg/day (for 80-100 kg body weights)

Broiler: 1360 mg/kg/day (for age one week after birth); 560 mg/kg/day (for age 8 weeks after birth)

Fish (trout): 900 mg/kg/day (for 1-2 g body weights); 160 mg/kg/day (for 40 g body weights)

The numerical figures of each animal above show the data at its small-size stage and at its large-size stage. At the stage between these stages, the intermediate numerical figures between these values are suggested.

For formulation of the inventive products into dosage forms for pharmaceutical products, sweeteners and flavor can be added to improve the taste and flavor. The dosage forms are not limited to them. Any dosage form may be satisfactory, including for example liquids such as suspensions and syrups. Even in this case, additionally, sweeteners and flavor can be added to improve the taste and flavor, as described above. If necessary, vitamin and inorganic matters may also be added thereto. The inventive products may be formulated into injections for intravenous administration. For producing these dosage forms, the techniques generally used in the pharmaceutical industry can be used for ready production thereof.

In case of foods or drinks, appropriate amounts of lysine can be added and mixed at the stage of or after the production of various foods or drinks, depending on the type and form of each of the foods or drinks. The inventive products can be prepared into foods or drinks supplemented with a higher amount of lysine. For example, lysine is preliminarily added to cereals at small contents of lysine, such as corn. When the resulting cereals are then incorporated as main diet, the onset of stress-induced diseases can significantly be suppressed. As the intake, lysine at preferably approximately 0.001 to 1.0 g/kg/body weight, more preferably approximately 0.01 to 0.5 g/kg/body weight daily is appropriate on a free form basis. More preferably, lysine is incorporated from the inventive products, so that the total lysine intake per day may be within the range of the numerical figures described above.

Feeds can be readily prepared according to the already known technique for lysine-supplemented feeds, more preferably by blending lysine set at a higher content level as described above.

Furthermore, it is effective to increase the intake of lysine in normal subjects or patients affected with stress-induced diseases or potential patients with such diseases, by allowing them ingest lysine in the forms of foods or drinks, medical foods or health foods containing lysine as the effective (active) ingredient. As described above, the inventive products are effective, when they are given before or during stress loading for the purpose of preventing thereof. The inventive products are particularly effective as preventive tools of stress-induced diseases due to lysine deficiency.

As described above, in an additional aspect, the invention relates to a method for suppressing stress(anti-stress method), including lysine incorporation (ingestion) in or administration to biological organism(s) (living body(ies)); and in a still additional aspect, the invention relates to a use of lysine in (for) anti-stress agents or production thereof. In these cases, lysine may be in the form of salt(s).

These inventions can be carried out readily, on the basis of the descriptions about the individual pharmaceutical compositions, the foods or drinks, the feeds and the like in accordance with the invention or the descriptions about the agent against (for opposing) stress-induced diseases in accordance with the invention, the after described Examples and the like, with reference to known art, if necessary.

Having generally described this invention, a further understanding can be obtained by reference to certain specific examples, which are provided herein for purposes of illustration only, and are not intended to be limiting unless otherwise specified.

### EXAMPLES

Herein, amino acids used in the examples are all in their L forms.

### Example 1

### Model for Assessing Anxiety Disorders

A low lysine diet was prepared, using wheat gluten at a reduced lysine content as the main raw material. The lysine content of the wheat gluten is about 1/4-fold the ideal lysine requirement, which is a lysine-containing diet that and maintain an increase of rat body weight. To prepare a lysine-added diet, lysine was added to the low lysine diet to reach a lysine concentration at the ideal requirement level of lysine. To ensure a uniform nitrogen source, lysine was added instead of glutamine, at an amount corresponding to the amount of glutamine). Table 1 shows the compositions of the low lysine diet and the lysine-added diet used in this experiment. After 2-week feeding on each diet, Wistar rats (male; age 5 weeks) were used (each group of 6 rats; n=6). Elevated T-maze test was done with reference to the method of Graeff, et al.

A T-maze was arranged at a height of 0.9 m above the ground surface so that one part may be a box while the remaining two parts may be in an open environment. First, the rats were placed in the box, and the rats “searching” actions thereof were observed through a television monitor. Differences in the time duration and number of their searching actions at such a simple elevated T-maze test was not observed between the rats on the lysine-added diet and the rats on the low lysine diet.

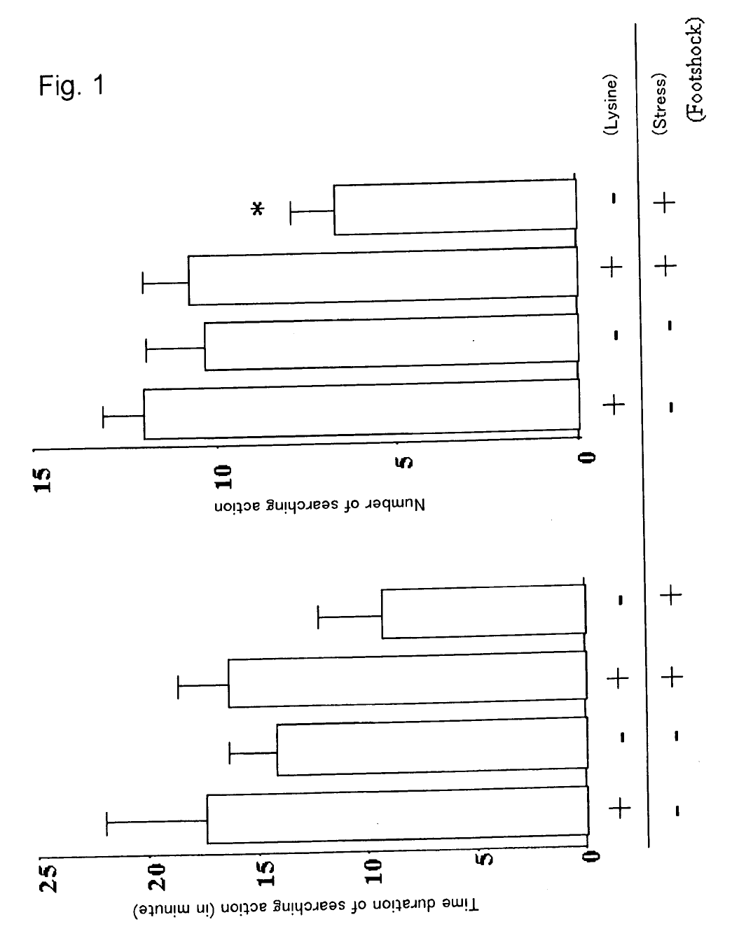

When mild stress (foot shock stress) was loaded immediately before the start of the experiment, however, the time duration and number of searching actions in the rats on the lysine-added diet were not decreased under observation. In contrast, rats on the low lysine diet had a significant reduction in time duration and number thereof (p<0.05) (see FIG. 1). In other words, their anxiety symptoms were exacerbated.

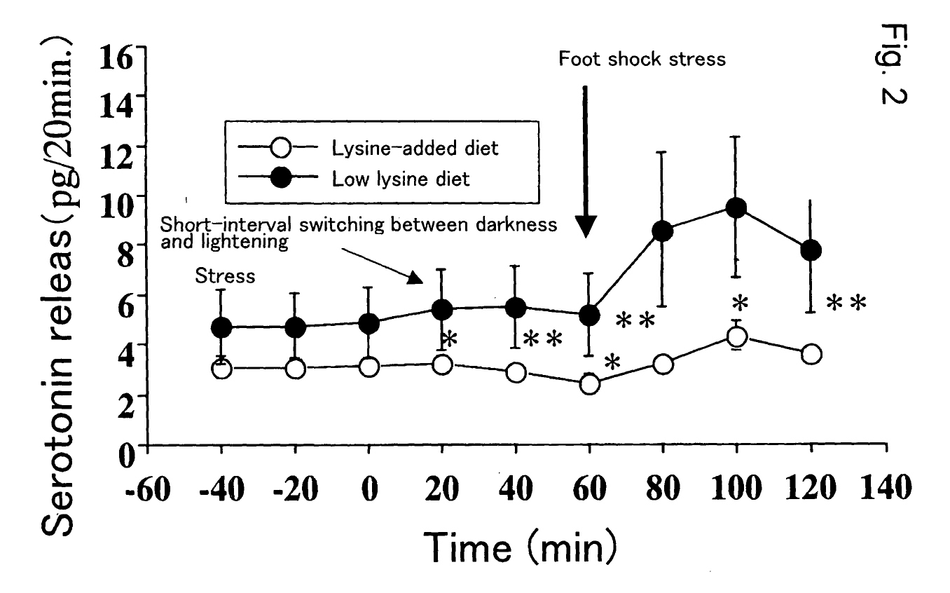

The serotonin concentration in the rat brain (amygdala) after foot shock was assayed over time by the microdialysis method. It was determined that the serotonin concentration in the rats on the low lysine diet was significantly increased, compared with the rats on the lysine-added diet (see FIG. 2). Amygdala functions as the center of affections such as emotion, while serotonin is generally believed to be one of the transmitters. These indicate that lysine deficiency enhances anxiety state, as taught by behavioral science and neurochemically. Thus, it is indicated that lysine supplement in diet can ameliorate such a state.

TABLE 1

| | Low lysine diet | Lysine-added diet |
|---|---|---|
| | (composition ratio in %) | (composition ratio in %) |
| Corn starch | 20.16 | 19.89 |
| Gluten Mix *1 | 28.07 | 28.07 |
| Pre Mix45 *2 | 45 | 45 |
| Vitamin E | 0.01 | 0.01 |
| Corn Oil | 5 | 5 |
| L-Lysine | 0 | 1.35 |
| L-Glutamine | 1.76 | 0.68 |
| Total | 100 | 100 |

*1 see Smriga, et al., J. Nutrition 130, 1641-1643, 2000
*2 (Composition) Starch at 79.6%, cellulose at 8.9%, inorganic mixture at 8.9%, vitamin mixture at 2.2% and choline Cl⁻ at 0.4%.

### Example 2

### Model of Irritable Colon Syndrome

Psychological stresses result in the activation of gastric motion, particularly bowel motion (i.e., motion for feces excretion). As many people can attest, once on a train or on a schedule prior to a presentation in an important meeting, the person may experience a sudden urge to rush to the toilet. While under more significant stress, the person may even experience diarrhea-like symptoms. People with severe such symptoms are called irritable colon syndrome and need therapeutic treatment.

It has been determined that the feces excretion motion of small animals, such as rats, is activated upon mild stress. Specifically, both the forefeet of a rat are fixed around its body with tape (wrap stress) to block spontaneous action. Then, the amount of feces is observed. This is called wrap stress resistant model (WRS) and is used for screening pharmaceutical products for the purpose of the therapeutic treatment of the irritable colon syndrome.

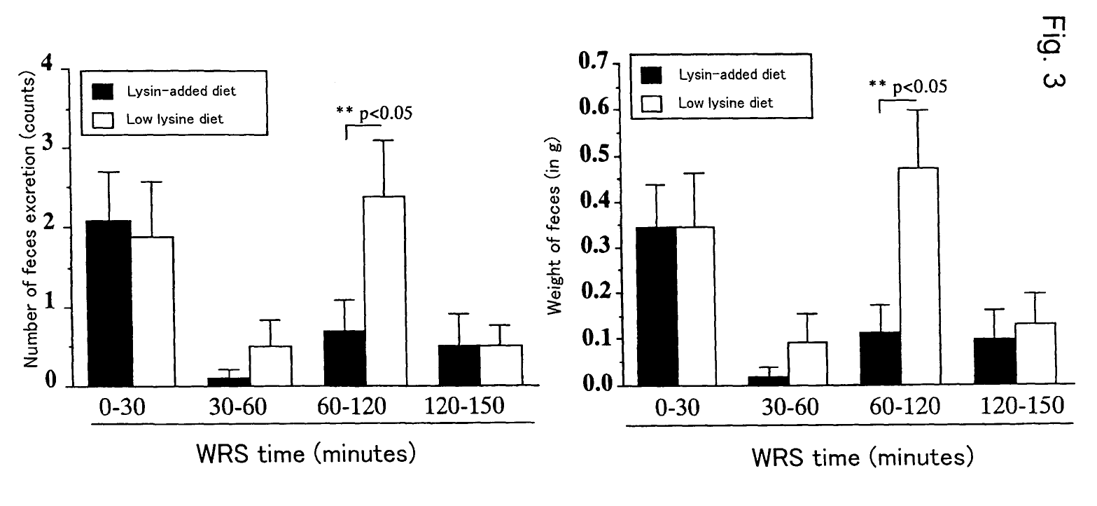

Wistar rats (male; age 5 weeks) were fed with the lysine-added diet and the low lysine diet (lysine deficient diet; see Table 1) for 2 weeks. In each group 10 animals were tested. WRS model was prepared by the method of Ito C. (J. Pharmacol. Exp. Ther., 1997, 280, 67-72) and Kishibayashi N., et al. (Jpn. J. Pharmacol., 1993, 63, 495-502). Specifically, the forefeet of the rats were fastened around its body with a cotton tape. The rat was then left in a feeding bracket cage. The excreted feces were collected at an interval of 10 minutes. FIG. 3 shows the results of the number of feces excretion and the weight of the excreted feces after collected and integral calculation at a 30-min interval, starting the loading of wrap stress to 150 minutes later. Both the rats on the lysine-added diet and the rats on the low lysine diet had peaks of their actions for feces excretion, within 30 minutes after the start of WRS. Subsequently, the actions were gentler with a peak indicating gentle action for feces excretion over one to 2 hours. Thus, their actions involved two-phase reactions. Between both of the groups, no difference was observed in terms of the number of feces excretion and the weight of excreted feces as caused by immediate stress-induced bowel motion as observed in the phase I. However, both the number of feces excretion and the weight of excreted feces due to gradual stress-induced bowel motion as observed in the phase II appearing in the delayed stage were significantly larger in the rats on the low lysine diet (p<0.05). Accordingly these data evince that lysine deficiency enhances feces excretion due to psychological stress, which is ameliorated by the intake of the lysine-added diet.

### Example 3

### Model of Stress-Induced Gastric Ulcer

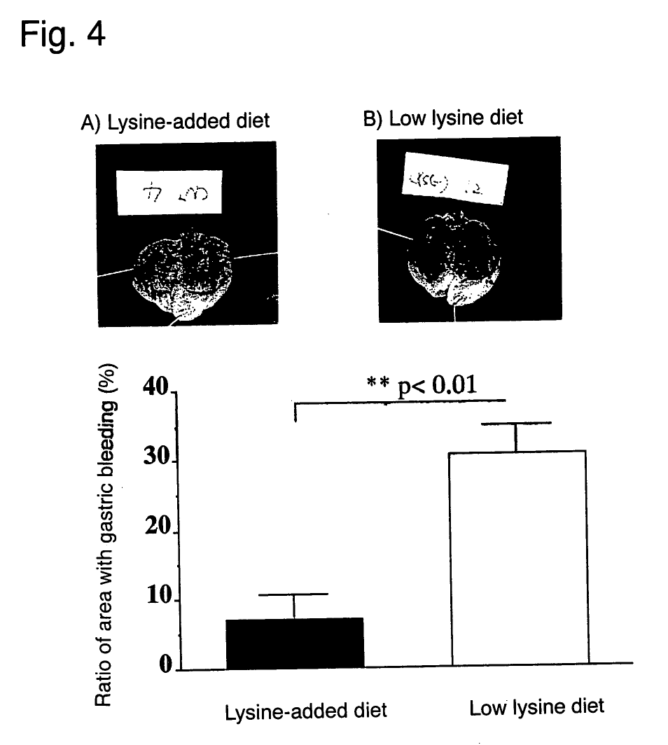

Rats were fed with the low lysine diet and the lysine-added diet supplemented for lysine deficiency (see Table 1) for 3 weeks. After starvation for 18 hours, the rats were placed in a stress gage (Natsume Seisakusho; KN-468) to immerse the rats in water (temperature of 22 to 25° C.) so as to soak their breast for 6 hours. Thereafter, a stomach was resected, to calculate the area with gastric bleeding, to examine the degree of gastric bleeding with the NHI image software. FIG. 4 shows the results obtained for the stress-induced gastric ulcer. Compared with the rats fed with the lysine-added diet, the rats fed with the low lysine diet had significantly increased the degree in areas of gastric bleeding (the ratio of the area of gastric bleeding to the total gastric area) due to the water-immersion restraint. A correlation between gastric bleeding and the incidence of gastric ulcer is observed in this model. Therefore, it can be concluded that adding lysine to diet can prevent stress-induced gastric ulcer occurring on the low lysine diet.

### Example 4

### Effect of Lysine-Added Diet in Model with Stress-Induced Appetite Loss

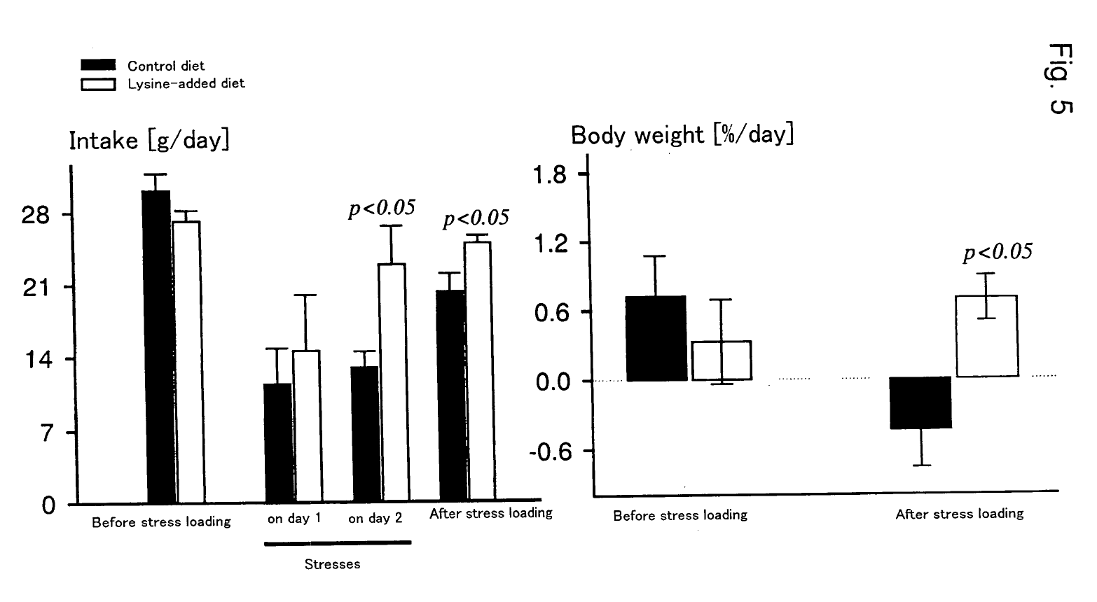

Wistar rats (male; age 13 weeks; about 400 g or so; N=16) were used at this experiment. Four days before the start of the experiment and throughout the stress experiment, the rats were fed with normal diet (control diet; lysine at 13.4 g/kg) and the lysine-added diet (27 g/kg). Under conditions of water ad libitum, feeding was done from 9:00 to 11:00. The rats were subjected to foot shock stress (1 mA/3 minutes; once per one hour at 19:00 to 7:00 on the next day) on day 4 of feeding; foot shock stress (1 mA/3 minutes, once per 2 hours at 19:00 to 7:00 on the next day) on day 5; and foot shock stress (1 mA/3 minutes, once at 7:00) on day 6. The changes in the total feed intake each day and the change of body weight over days were measured. The results are shown in FIG. 5.

Table 2 below shows the compositions of the control diet and the lysine-added diet used herein.

TABLE 2

| | Control diet (composition | Lysine-added diet |
|---|---|---|
| | ratio in %) | (composition ratio in %) |
| Corn starch | 19.89 | 18.54 |
| Gluten Mix *1 | 28.07 | 28.07 |
| Pre Mix45 *2 | 45 | 45 |
| Vitamin E | 0.01 | 0.01 |
| Corn Oil | 5 | 5 |
| L-Lysine | 1.35 | 2.70 |
| L-Glutamine | 0.68 | 0.68 |
| Total | 100 | 100 |

*1 see Smriga, et al., J. Nutrition 130, 1641-1643, 2000
*2 (Composition) Starch at 79.6%, cellulose at 8.9%, inorganic mixture at 8.9%, vitamin mixture at 2.2% and choline CV⁻ at 0.4%.

The above results indicate that the lysine administration before stress loading allows for the exertion of the preventive effect and also suggest the effect of lysine administration on the amelioration and therapeutic treatment of stress-induced diseases after their onset. In other words, lysine is more preferably administered before stress loading from the prophylactic standpoint. After the onset, lysine administration can expedite recovery. Even in this case, lysine administration before stress loading can enhance the effect more.

### Example 5

### Stress-Induced Gastric Ulcer Experiment

To assess the effects of lysine administration on stress-induced gastric ulcers, 7-week Wistar rats (male; Nippon Charles-River) were used. For 7 days, normal diet (CRF-1; Oriental Yeast) was fed ad libitum to the rats, for preliminary feeding. For the stress experiment, rats were divided into the following three groups for feeding for another 3 days under starvation conditions: I) a control group (N=8) fed with pure water; 2) a group (N=8) given with an aqueous lysine solution (40 mg/mL); and 3) a group (N=4) given with an aqueous mixture solution of lysine glutamate salt (40 mg/mL) and arginine (40 mg/mL). The aqueous solutions were given to the individual groups in such a manner that dosing of 5 mL of each solution was enforced with an oral probe, once daily at 9:00 in the morning.

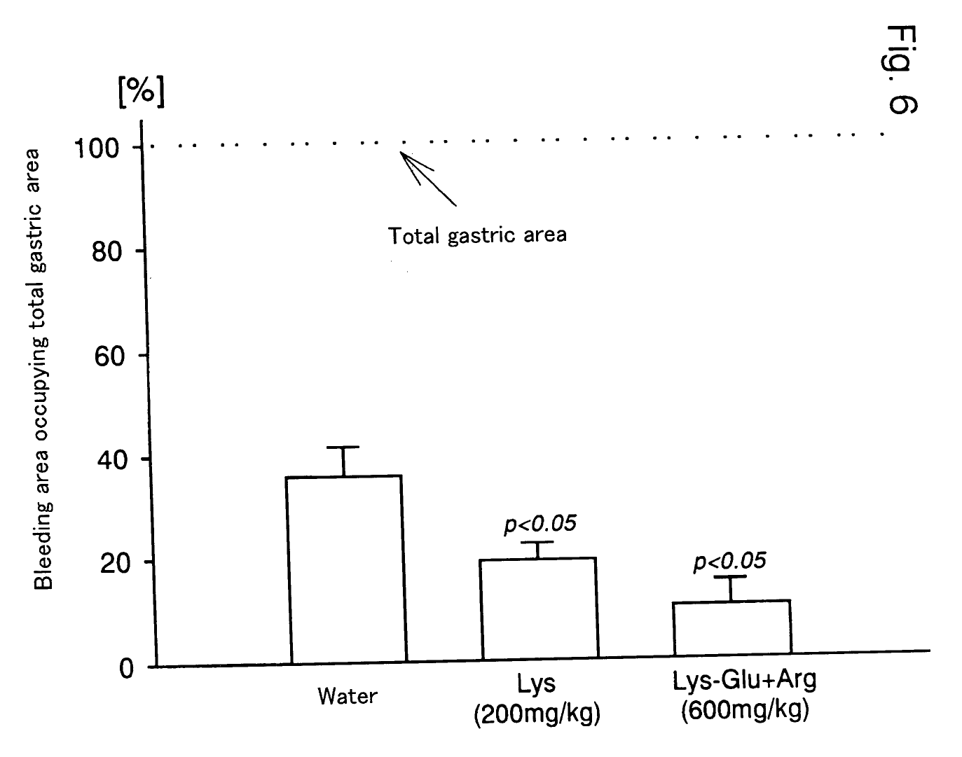

During the term, water was fed ad libitum. On day 4, the rats were placed in a stress gage (Natsume Seisakusho; KN-468) as in Example 3, to immerse the rats in water (temperature of 22 to 25° C.) so as to soak their breast for 5 hours to induce stress-induced gastric ulcer. The results are shown in FIG. 6. Compared with the control group fed with pure water, the aqueous lysine solution group and the group fed with the aqueous mixture solution of lysine glutamate salt and arginine was observed to have significant reduction of the areas of gastric bleeding. These results suggest that addition of lysine in the form of aqueous solution can prevent stress-induced diseases even under feeding with normal diet.

### Example 6

### Effect of Lysine and Lysine-Containing Amino Acid Dosage Form at Elevated T-Maze Test

Method

Seven-week Wistar rats (male, Nippon Charles-River) were used. For 7 days, the normal diet (CRF-1; Oriental Yeast) was fed ad libitum to the rats, for preliminary feeding. For the stress experiment, the rats were divided into the following three groups for feeding for another 3 days under starvation conditions: 1) a group (N=14) fed with an aqueous glutamine solution (120 mg/mL); 2) a group (N=14) given with an aqueous lysine solution (120 mg/mL); and 3) a group (N=14) given with an aqueous mixture solution of lysine glutamate salt (120 mg/mL) and arginine (120 mg/mL). The aqueous solutions were given to the individual groups in such a manner that dosing of 5 mL of each solution was enforced with an oral probe, once daily at 9:00 in the morning. During the term, water was fed ad libitum. On day 4, the rats were placed in a stress gage (Natsume Seisakusho; KN-468) to immerse the rats in water (temperature of 22 to 25° C.) so as to soak their breast for 4 hours for stress loading. Thereafter, elevated T-maze (see Example 1) test was done, for counting the time duration for searching action.

Results

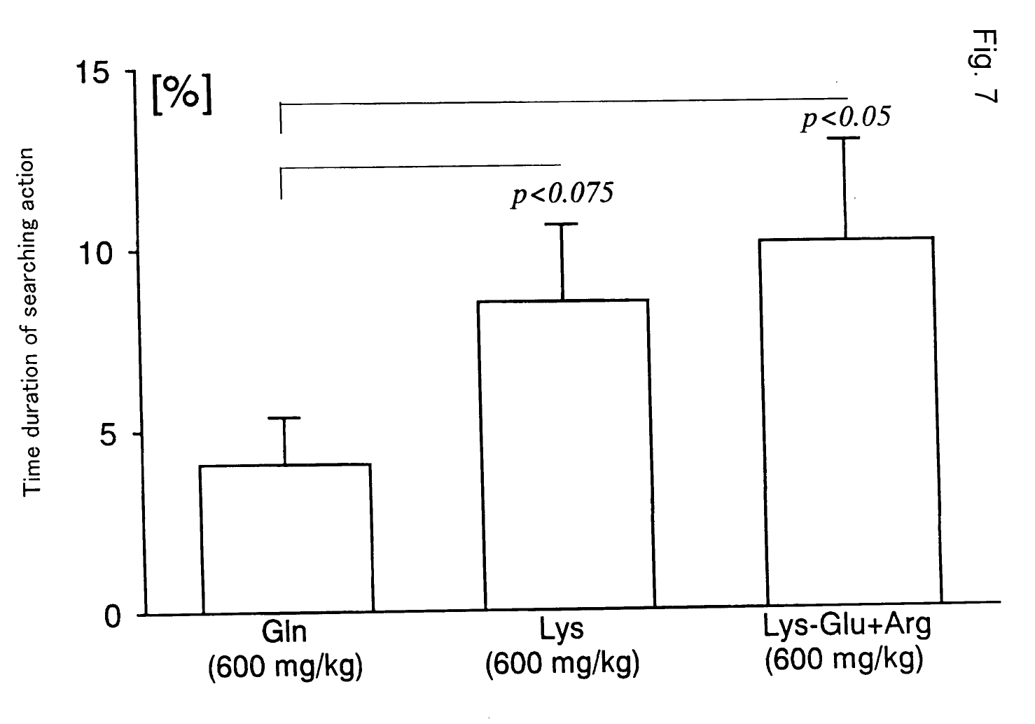

The results are shown in FIG. 7. Compared with the glutamine group, the lysine group and the group fed with the aqueous mixture solution of lysine glutamate salt and arginine had significantly prolonged time duration for searching action. Additionally, to evaluate the effect of glutamine, a group fed with physiological saline and a group fed with glutamine was prepared. No significant difference was observed between the two groups. The results described above verified that the dosage form containing lysine alone or the lysine-containing amino acid dosage form prevented anxiety and hypersensitivity after stress loading.

### Example 7

### Stress from Feeding at High Density During Fishery and Culture

A report tells that the lysine requirement for mojyako (the other name of young yellow tail) according to nutrition science is at 1.78% in dried feed (see T. Ruchimat et al., Aquaculture 158 (1997), 331-339).

Generally, lysine in an amount exceeding the requirement even when given cannot improve the result of feeding. However, in a circumstance under stress loading, lysine when administered at a level excessive from the standpoint of nutrition science can prevent the reduction of feed intake due to stress, which consequently improves the feeding results. The following experiment is one of the examples.

Test feeds were prepared by adding lysine hydrochloride salt at the ratio shown in Table 3 below to a commercially available mashed feed for mojyako. The lysine content in the dry feed is thereby at 4.8%, which is far above the dietetic requirement (1.78%). For comparison, further, a feed was prepared by adding arginine hydrochloride salt at the same level instead of lysine hydrochloride salt, for use at the test.

As test fish, each group of 40 mojyako fishes of mean fish body weight of about 35 g were placed in one 800-L water tank, to which the feeds were fed two times daily at 8:00 in the morning and 16:00 in the afternoon to satiation, for a total of 4 weeks. During the term, their body weights were measured weekly. Herein, feeding was not done at 16:00 in the afternoon on the day before the day for body weight measurement.

TABLE 3
Composition of feed blends

| Test feeds | Control | Arginine added | Lysine added |
|---|---|---|---|
| Mashed feed | 860 | 820 | 820 |
| Fish oil | 140 | 140 | 140 |
| Water | 400 | 400 | 400 |
| Arginine HCl | 0 | 40 | 0 |
| Lysine HCl | 0 | 0 | 40 |
| Moisture (%) | 32.4 | 30.8 | 31.7 |
| Protein (%) | 25.7 | 28.5 | 28.2 |
| Lysine (% in dry | 1.8 | 1.8 | 4.8 |
| weight) | | | |
| Lipid (%) | 15.4 | 14.8 | 14.7 |
| Ashes (%) | 7.91 | 7.89 | 7.78 |

The feeding results are shown in Table 4.

TABLE 4
Growth results

| Test lots | | Control | Arginine | Lysine |
|---|---|---|---|---|
| Test duration | | 28 | 28 | 28 |
| (day) | | | | |
| Feeding | | 24 | 24 | 24 |
| duration (day) | | | | |
| Survival rate | | 100 | 100 | 100 |
| (%) | | | | |
| Mean body | at start | 35.1 | 35.5 | 34.8 |
| weight | on 1 week | 45.5 | 45.7 | 46.4 |
| | on 2 week | 62.7 | 61.5 | 63.7 |
| | on 3 week | 79.9 | 77.6 | 84.2 |
| | on 4 week | 95.7 | 98.5 | 108.9 |
| | (final) | | | |
| Daily | | 3.31 | 3.36 | 3.68 |
| increment (%) | | | | |
| Total feed | | 3034 | 3072 | 3452 |
| intake (g) | | | | |
| Weekly | for 0-1 week | 18.6 | 18.7 | 18.4 |
| feed intake (g) | for 1-2 week | 37.0 | 36.7 | 38.3 |
| | for 2-3 week | 54.3 | 54.5 | 59.9 |
| | for 3-4 week | 74.0 | 76.8 | 86.3 |
| Daily feeding | | 4.09 | 4.09 | 4.29 |
| ratio (%) | | | | |
| Feed efficiency | | 82.9 | 82.0 | 85.8 |
| (%) | | | | |

It is assumed that higher stress is loaded to such feeding in water tanks as in this feeding test, compared with normal feeding in seawater. Further, it is also assumed that as the fish body weight increases during the feeding test, stress due to such highly densified feeding in the same volume of a water tank will be intensified. In the lysine lot, the feed intake was larger than those of the remaining experimental lots over the 3 week to 4 week, involving higher body weight increment (at the termination of the test, the increment was 17%, compared with the control).

Because such effect could not be obtained in the arginine lot, the effect is believed to be inherent to lysine under stress conditions.

### Example 8

### Stress on Broiler from Feeding at High Density

Experimental Method

Broiler (species: Arbor Acres) was fed under very hot conditions. The compositions of the feeds are as shown in Tables 5 and 6.

TABLE 5

| | Normal diet | Lysine-added | (Lysine + arginine)- |
|---|---|---|---|
| | [kcal/kg] | feed [kcal/kg] | added feed [kcal/kg] |
| Crude protein | 18 | 18 | 18 |
| Crude fat | 6.2 | 6.2 | 6.2 |
| Linoleic acid | 1.91 | 1.91 | 1.91 |
| Crude fiber | 2.38 | 2.38 | 2.38 |
| L-Lysine | 0.85 | 1.7 | 1.7 |
| L-Arginine | 1.18 | 1.18 | 2.36 |
| L-Methionine | 0.32 | 0.32 | 0.32 |
| L-Cysteine | 0.39 | 0.39 | 0.39 |
| L-Threonine | 0.71 | 0.71 | 0.71 |
| Tryptophan | 0.16 | 0.16 | 0.16 |
| L-Serine | 1.62 | 1.62 | 1.62 |
| L-Isoleucine | 0.75 | 0.75 | 0.75 |
| L-Leucine | 1.73 | 1.73 | 1.73 |
| L-Valine | 0.92 | 0.92 | 0.92 |
| L-Glycine | 0.58 | 0.58 | 0.58 |
| Calcium | 0.9 | 0.9 | 0.9 |
| Phosphorus | 0.59 | 0.59 | 0.59 |
| Non-phytin | 0.4 | 0.4 | 0.4 |
| phosphorus | | | |
| Sodium chloride | 0.4 | 0.4 | 0.4 |

TABLE 6

| | Control | Lysine added | (Lysine + Arginine) |
|---|---|---|---|
| | [%] | [%] | added [%] |
| Corn | 67.40 | 67.40 | 67.40 |
| Corn starch | 3.00 | 2.42 | 1.82 |
| Soy bean bran | 14.20 | 14.20 | 14.20 |
| Corn gluten meal | 2.50 | 2.50 | 2.50 |
| Fish powder | 1.00 | 1.00 | 1.00 |
| Feather meal | 4.00 | 4.00 | |
| Palm oil | 3.20 | 3.20 | 3.20 |
| Hydrochloric acid | 0.59 | 1.17 | 1.17 |
| L-lysine | | | |
| DL-Methionine | 0.07 | 0.07 | 0.07 |
| L-Arginine | 0.60 | 0.60 | 1.20 |
| L-Tryptophan | 0.01 | 0.01 | 0.01 |
| Dibasic calcium | 1.15 | 1.15 | 1.15 |
| phosphate | | | |
| (CaHPO₄) | | | |
| Calcium carbonate | 1.75 | 1.75 | 1.75 |
| Edible salt | 0.28 | 0.28 | 0.28 |
| Premix | 0.25 | 0.25 | 0.25 |
| Total | 100.0 | 100.0 | 100.0 |

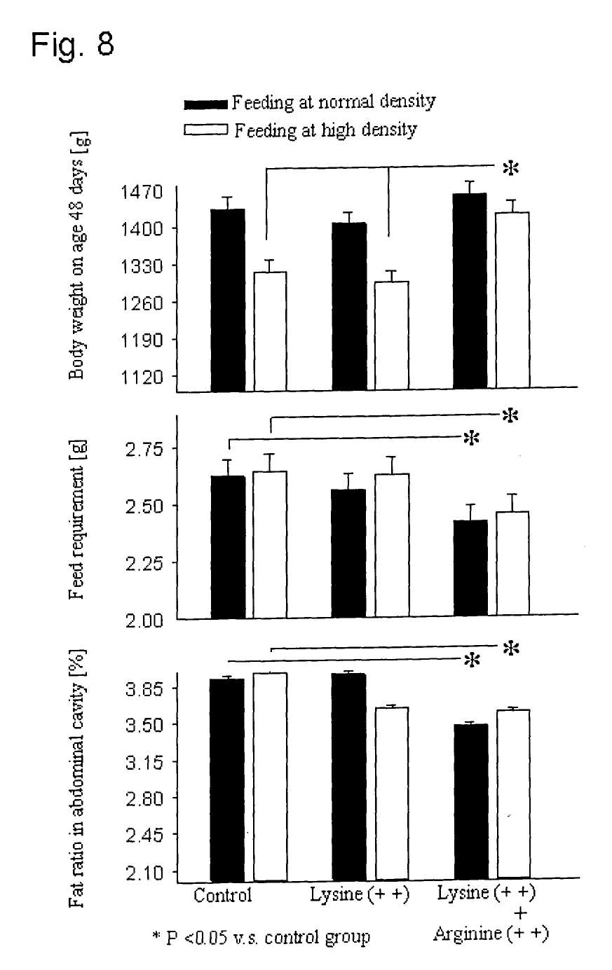

The feeding density was 8 chickens/1 m² (normal density) and 12 chickens/1 m² (high density) in two lots; the feeds were the following three groups: Feed 1=normal feed; Feed 2=normal feed+lysine (the lysine content 2-fold that in Feed 1); or Feed 3=normal feed+lysine+arginine (the lysine content and the arginine content were individually 2-fold those in Feed 1). A total of 6 experimental examples were set as combinations of the feeding densities and the feeds. The test was started on age 21 days (the mean body weight then was 722 g). The feeding results (body weight increment, feed requirement) on age 48 days were compared thereto. On the age 48 days, the chickens were sacrificed to death, for the evaluation of their meat quality (fat ratio in abdominal cavity). The feed requirement was expressed by the feed amount required for the increment of 1 g/body weight, as calculated by dividing the total feed intake by the body weight increment. The feeding results are shown in FIG. 8.

Results

In the normal feed group, the reduction of the feed intake and body weight increment in the high density feeding lot was observed, which may be ascribed to the stress from the feeding at such high density. In the lysine-added feed group, no improvement of body weight and feed requirement was observed in the normal density feeding lot and the high density feeding lot. However, the fat ratio in abdominal cavity was likely to decrease in the group. In the (lysine+arginine)-added feed group, no body weight increase was observed under feeding at normal density. The results may be due to the reduction of the stress from the feeding at high density, by the feeding of the (lysine+arginine) feed. By comparison in terms of meat quality, on the other hand, the fat ratio in abdominal cavity and the feed requirement were significantly reduced in the (lysine+arginine)-added feed group, so that the improvement of meat quality as well as the improvement of feeding efficiency was observed. The results of these experiments indicate that the amino acid dosage form (lysine+arginine) containing lysine apparently can improve the feeding efficiencies (body weight increase, reduction of feed requirement and reduction of fat ratio in abdominal cavity) of broilers under very hot conditions.

For broilers, the antagonistic action between lysine and arginine is known. A report tells that the ratio of lysine and arginine in feeds should be controlled at an appropriate level. At the experiments, thus, arginine was added in the lysine lot, so that the ratio of lysine to arginine might be constant. In other words, it is known that the ratio of arginine/lysine in the amino acid composition of feeds is very important at the growth stage of broilers for the growth thereof and that a smaller ratio thereof more readily induces growth inhibition. It is reported that the action is prominently great in broiler, compared with mammalian cases, such as rat, dog and pig (see J. Am. Coll. Nutrition 16, 1997, 7-21; J. Nutrition 115, 1985, 743-752; FASEB Special Publications Office, pp 22, 59, 1992). At the present experiment, at least the ratio of arginine/lysine in the feed is lowered via the loading of lysine alone, but no growth inhibition is observed. This may be ascribed to the results of the masking of the body weight increase due to the anti-stress action of lysine with the weight decrease through the reduction of the ratio arginine/lysine, so that it can be said that the effect can substantially be observed.

As described above, it was confirmed that the problem of poor feeding results of broiler due to the stress from the high density feeding could be solved by the addition of lysine/arginine.

The results of the above Examples indicate that lysine is effective as an agent against (for opposing) stress-induced diseases, particularly as an agent for preventing stress-induced diseases. It is thus understood that lysine can be used widely in pharmaceutical compositions, foods or drinks or feeds or the like so as to obtain the effect. Additionally, it is understood that the combined use of other specific amino acids, for example glutamic acid and arginine can further enhance the effect.

Numerous modifications and variations on the present invention are possible in light of the above teachings. It is, therefore, to be understood that within the scope of the accompanying claims, the invention may be practiced otherwise than as specifically described herein.

### ADVANTAGES OF THE INVENTION

In accordance with the present invention, pharmaceutical products (pharmaceutical compositions), foods or drinks or feed effective for the prevention, amelioration and therapeutic treatment and the like of stress-induced diseases. The pharmaceutical products of the present invention employing lysine as the active (effective) ingredient are particularly effective for the prevention of stress-induced diseases. Additionally, the present invention provides a lysine-containing agent against stress-induced diseases. The present invention further provides a method for suppressing stress (anti-stress method) and a use of lysine for these agents or pharmaceutical drugs or the like, or production thereof.

Thus, the invention is applicable widely in the fields of pharmaceutical products, foods, feeds, clinical practices and the like, and is therefore very useful industrially.

1. A method of preventing, ameliorating, progress blocking, or therapeutically treating one or more stress-induced diseases comprising administering to a subject in need thereof an effective amount of a pharmaceutical composition comprising lysine.

2. The method of claim 1, wherein the lysine is L-lysine.

3. The method of claim 1, wherein the lysine is in a free form.

4. The method of claim 1, wherein the lysine is in a salt form.

5. The method of claim 1, wherein said pharmaceutical composition is selected from the group consisting of a food, a drink, or a feed.

6. The method of claim 1, wherein said pharmaceutical composition further comprises one or more additional amino acids.

7. The method of claim 6, wherein said one or more additional amino acids are in a free form.

8. The method of claim 6, wherein said one or more additional amino acids are in a salt form.

9. The method of claim 6, wherein said one or more additional amino acids are individually in a free or salt form.

10. The method of claim 6, wherein at least one of said one or more additional amino acids is selected from the group consisting of arginine, glutamic acid, and aspartic acid.

11. The method of claim 1, wherein the lysine is in a salt form salt with glutamic acid or aspartic acid.

12. The method of claim 1, wherein the lysine is in a salt form salt with glutamic acid and which contains arginine.

13. The method of claim 1, wherein said administering entails providing to said subject the pharmaceutical composition prior to or during stress loading.

14. The method of claim 1, wherein said subject in need thereof suffers from lysine deficiency.

15. The method of claim 1, wherein said stress-induced diseases include one or more diseases selected from the group consisting of anxiety disorders, psychosomatic disorders, dissociated disorders, emotion disorders, abnormal gastric motion, irritable colon syndrome, irritable bowel syndrome, and gastric ulcer.

16. The method of claim 1, wherein said effective amount is 0.001 to 13 g/kg/body weight daily on a free lysine form basis.

17. The method of claim 1, wherein said effective amount is such that the total intake of lysine is 0.001 to 13 g/kg/body weight daily on a free lysine form basis.

18. The method of claim 1, wherein said pharmaceutical composition contains lysine at 90 to 0.1% by weight on a free form basis.

19. The method of claim 1, wherein said pharmaceutical composition contains a lysine content of 1.1- to 3.0-fold the recommended nutritious requirement of lysine.

20. The method of claim 1, wherein said subject in need thereof is an animal selected from the group consisting of human, cattle, pig, chicken, and culture fish.

21. The method of claim 20, wherein said subject in need thereof is a human.

22. The method of claim 1, wherein said pharmaceutical composition further comprises inorganic matter.

23. The method of claim 1, wherein said pharmaceutical composition further comprises a pharmaceutically acceptable carrier or excipient.

24. A pharmaceutical composition comprising lysine and a pharmaceutically acceptable carrier or excipient.

25. The pharmaceutical composition of claim 24, wherein lysine is at a concentration of 90 to 0.1% by weight on a free form basis.

26. The pharmaceutical composition of claim 24, wherein said pharmaceutical composition further comprises inorganic matter.
